(19) United States
(12) Patent Application Publication
(10) Pub. No.: US 2013/0310307 A1
(43) Pub. Date: Nov. 21, 2013

(54) METHODS AND COMPOSITIONS FOR TREATMENT OF MULTIDRUG-RESISTANT BACTERIAL AND FUNGAL INFECTIONS

(71) Applicant: LOS ANGELES BIOMEDICAL RESEARCH INSTITUTE AT HARBOR-UCLA MEDICAL CENTER, TORRANCE, CA (US)

(72) Inventors: Brad J. SPELLBERG, Rancho Palos Verdes, CA (US); Lin LIN, Rancho Palos Verdes, CA (US)

(73) Assignee: LOS ANGELES BIOMEDICAL RESEARCH INSTITUTE AT HARBOR-UCLA MEDICAL CENTER, TORRANCE, CA (US)

(21) Appl. No.: 13/801,324

(22) Filed: Mar. 13, 2013

Related U.S. Application Data
(60) Provisional application No. 61/638,947, filed on Apr. 26, 2012.

(51) Int. Cl. A61K 38/17

(52) U.S. Cl. CPC A61K 38/1709
USPC 514/2.7; 514/2.3; 514/2.8; 514/3.3; 514/3.1; 514/3.4

(57) ABSTRACT

The invention provides a method of killing an infectious microbe by administering an effective amount of transferrin to an individual having a microbial infection, wherein the transferrin has microbicidal activity, thereby reducing survival of the infectious microbe in the individual. The invention also provides a method of prophylactically treating an individual to decrease the likelihood of contracting a microbial infection, comprising administering an effective amount of transferrin to an individual, wherein the transferrin has microbicidal activity, thereby decreasing the likelihood that the individual will contract a microbial infection. The invention still further provides a method of treating septicemia by administering an effective amount of transferrin to an individual in need thereof, thereby treating the individual.

This application claims the benefit of priority of U.S. Provisional application Ser. No. 61/638,947, filed Apr. 26, 2012, the entire contents of which is incorporated herein by reference.

This invention was made with government support under grant number R01 AI081719-01A1 awarded by the National Institutes of Health. The government has certain rights in the invention.

The present invention relates generally to microbial infections, and more specifically to methods of treating microbial infections.

Infectious diseases remain among the leading causes of morbidity and mortality on our planet. The development of resistance in microbes—bacterial, viral, or parasites—to therapeutics is neither surprising nor new. However, the scope and scale of this phenomenon is an ever-increasing multinational public health crisis as drug resistance accumulates and accelerates over space and time. Today some strains of bacteria and viruses are resistant to all but a single drug, and some may soon have no effective treatments left in the “medicine chest.” The disease burden from multidrug-resistant strains of organisms causing AIDS, tuberculosis, gonorrhea, malaria, influenza, pneumonia, and diarrhea is being felt in both the developed and the developing worlds alike. (see Antibiotic Resistance: Implications for Global Health and Novel Intervention Strategies, Institute of Medicine (US) Forum on Microbial Threats. Washington (D.C.); National Academies Press (2010)).

Antimicrobial resistance most commonly refers to infectious microbes that have acquired the ability to survive exposures to clinically relevant concentrations of drugs that would kill otherwise sensitive organisms of the same strain. The phrase is also used to describe any pathogen that is less susceptible than its counterparts to a specific antimicrobial compound, or combination thereof. Resistance manifests as a gradient based on genotypic and phenotypic variation within natural microbial populations, and even microbes with low levels of resistance may play a role in propagating resistance within the microbial community as a whole.

Pathogens resistant to multiple antibacterial agents, while initially associated with the clinical treatment of infectious diseases in humans and animals, are increasingly found outside the healthcare setting. Therapeutic options for these so-called community-acquired pathogens, such as methicillin-resistant Staphylococcus aureus (MRSA) are extremely limited, as are prospects for the development of the next generation of antimicrobial drugs.

Thus, there exists a need to develop therapies that provide effective treatment of drug resistant microbes. The present invention satisfies this need and provides related advantages as well.

### SUMMARY OF INVENTION

The invention provides methods of killing an infectious microbe by administering an effective amount of transferrin to an individual having a microbial infection, wherein the transferrin has microbicidal activity, thereby reducing survival of the infectious microbe in the individual. The invention also provides methods of prophylactically treating an individual to decrease the likelihood of contracting a microbial infection, comprising administering an effective amount of transferrin to an individual, wherein the transferrin has microbicidal activity, thereby decreasing the likelihood that the individual will contract a microbial infection. The invention still further provides a method of treating septicemia by administering an effective amount of transferrin to an individual having septicemia, thereby treating the individual.

### BRIEF DESCRIPTION OF THE DRAWINGS

FIGS. 1A-1C. A biochemical fraction in stem cell conditioned media had broad antimicrobial effects. A) Killing of bacteria or fungi after 1 hour in cell culture media harvested after 5 days of mouse embryonic stem cell (MES) culture. B) HPLC was used to identify fractions that were distinct between conditioned and control culture media (DMEM). Fraction 79 was clearly different. C) HPLC-purified fraction 79 recapitulated conditioned media microbicidal effects.

FIGS. 2A-2C. Transferrin in conditioned media had broad antimicrobial effects. A) Silver-stained PAGE gel of Fraction 79 revealed a single band distinguishing conditioned from control media. B) After identification by MALDI-TOF of transferring in the Fraction 79 band from conditioned media, ELISA confirmed the presence of higher quantities of transferrin in conditioned than control media).C) Microbial killing by recombinant transferrin.

FIGS. 3A-3C. Transferrin mediated the microbicidal effects of conditioned media. Kill assays with conditioned media were repeated in the presence of anti-transferrin monoclonal antibody (A) or after transfection of stem cells with anti-transferrin siRNA constructs that suppressed Trf gene expression (B) and blocked microbicidal effects (C).

FIGS. 4A-5C. Time-kill curves show microbistatic effects of rhTransferrin. Bacterial and fungal organisms, including S. aureus (A), A. baumannii (B), and C. albicans (C) were inoculated and grown in the presence of 0.6 μg/mL, 6 μg/mL or 60 μg/mL of transferrin and compared to untreated controls. Organism density was assayed by serial sampling.

FIGS. 5A-4C. Human recombinant transferrin in vitro and in vivo. A) Kill assays were repeated using human recombinant transferrin (instead of murine transferrin). B) Human recombinant transferrin administered to mice (n=2 per group) in a small, preliminary pilot study confirmed that 3 mg/kg/d could be delivered safely. C) Mice (n=5 per group) were treated with 30, 90 and 270 mg/kg iv of rhTransferrin once daily for total 3 days.

FIGS. 6A and 6B. Survival curves for infected mice show the antimicrobial effects of transferrin. A) Balb/c mice (n=10 per group) infected with either S. aureus or C. albicans, or C3H/FeJ mice (n=10 per group) infected with A. baumannii, were treated with human transferrin (90 mg/kg/d×4 d). B) Balb/c mice (n=10 per group) were infected with C. albicans, and were treated with either human transferrin alone, as described in (A), or human transferrin and FeCl₃ (0.4 mg/kb) to saturate transferrin with free iron. Placebo indicates untreated infected mice. * indicates p<0.05 vs. placebo.

### DETAILED DESCRIPTION OF THE INVENTION

The present invention provides methods for treating microbial infections and is particularly useful for treating multi-drug resistant (antibiotic resistant) microbial infections. The ongoing crisis of antibiotic resistance demands new methods to prevent and treat infections caused by highly resistant organisms. While new antibiotics are critically needed, ultimately resistance often develops to any new antibiotic developed. As described herein, recombinant transferrin has been found to have microbicidal activity against bacterial and fungal pathogens. Further as disclosed herein, studies found that recombinant human transferrin (rhTransferrin) improved survival of mice given otherwise lethal S. aureus and C. albicans infection. While transferrin has long been known to sequester iron in serum, recombinant transferrin has not previously been shown to be effective at killing microbes in vitro or for treating infection in vivo. Based on the broad spectrum activity of transferrin described herein, the fact that it is a host protein and therefore likely to be safe to administer to humans, and the fact that it is already commercially available in good manufacturing practice (GMP)-grade form, transferrin provides an additional therapeutic agent to combat microbial infections. The invention therefore provides a new use of transferrin as an antimicrobial, microbicidal agent.

As used herein, the term “transferrin” refers to a plasma protein involved in iron transport through blood. Transferrin is a protein of about 80,000 daltons that binds iron and functions in iron transport (reviewed in Garrick, Genes Nutr. 6:45-54 (2011); Wang and Pantopoulos, Biochem. J. 434:365-381 (2011); Zhang and Enns, Hematology Am. Soc. Hematol. Educ. Program 207-214 (2009) (PMID: 20008200); Wally and Buchanan, Biometals 20:249-262 (2007); Thorstensen and Romslo, Biochem. J. 271:1-10 (1990)). Transferrin is a member of a family of proteins that bind free iron in the blood and bodily fluids. Transferrin is found in serum and functions to deliver iron to cells via a receptor-mediated endocytotic process as well as to remove toxic free iron from the blood. Transferrin is well known to those skilled in the art, and human transferrin is commercially available purified from serum or expressed recombinantly (Novozyme, Bagsvaerd Denmark; Sigma-Aldrich, St. Louis Mo.; Athens Research & Technology, Inc., Athens, Ga.; ProSpec-Tany TechnoGene Ltd., Rehovot Israel).

As used herein, the term “infection” refers to the multiplication of a parasitic organism within the body. Infection is the invasion of the host by microorganisms, which then multiply in close association with the host's tissues. The term infection specifically excludes the multiplication of normal flora such as that found on the skin, intestinal tract, and/or other parts of the body. However, as described in more detail below, it is understood that microorganisms considered to be part of the normal flora can, under certain circumstances, become infectious. Under such circumstances, such a microorganism would be considered to be capable of causing an infection. Those skilled in the art will readily understand the meaning of an infection as used herein.

As used herein, the term “septicemia” refers to a systemic (bodywide) illness that is due to invasion of the bloodstream by a pathogenic microbe. The invasion can come from a local seat of infection. The symptoms of septicemia include chills, fever and exhaustion. Septicemia is also known in the art as blood poising or septic fever. Septicemia can also lead to a related medical condition called sepsis. “Sepsis” refers to a medical condition characterized by a whole-body inflammatory state. Sepsis is caused by a host organism's immune system responding to an infection caused by, for example, bacteria, fungi, viruses, or parasites in the blood, urinary tract, lungs, skin, or other tissues. Common symptoms of sepsis include those related to an infection, and can be accompanied by high fevers, hot skin, flushed skin, elevated heart rate, hyperventilation, altered mental status, swelling, and low blood pressure.

Antimicrobial activity can be microbicidal or microbistatic activity. As used herein, “microbicidal” refers to the activity of an agent to kill a microorganism. Such a microbicidal agent is destructive to the microbe and can also be referred to as a germicide or antiseptic. A microbicidal agent would therefore have microbicidal activity that destroys the microorganism or otherwise prevents the microorganism from being able to replicate. Thus, the survival of a microorganism is reduced by killing or irreversibly damaging it. Antimicrobial activity that is “microbistatic” refers to the ability of an agent to reduce or inhibit the growth or proliferative ability of a target microorganism without killing it. Thus, a microbistatic agent inhibits the growth of a microorganism, whereas growth of the microorganism can generally be restored upon removal of the microbistatic agent. This contrasts with a microbicidal agent, where the microorganism is destroyed or irreversibly damaged such that it cannot grow. Methods for assessing microbicidal or micribistatic activity of an agent are well known to those skilled in the art.

As used herein, the terms “microbe,” “microbial,” “microbial organism” or “microorganism” refer to an organism that exists as a microscopic cell. The term encompasses prokaryotic or eukaryotic cells, in particular single cell eukaryotic organisms, or organisms having a microscopic size. A microbe includes bacteria as well as eukaryotic microorganisms such as fungi, including yeast.

As used herein, the term “infectious microbe” refers to a microbe that is capable of infecting an individual, and includes, for example, pathogens. An infectious microbe is understood to exclude the normal flora of an individual. However, it is also understood that an infectious microbe can include a microbe that is normally not infectious in an individual but has acquired an infectious capability. Such an organism or infection can also be referred to as an opportunistic organism or infection. For example, a microbe that comprises the normal flora of an individual can acquire an infectious capability or can become infectious in an individual with a compromised immune system. Such an individual can acquire a comprised immune system through an infection or illness or administration of immunosuppressive drugs, thereby permitting a microbe that is generally not infectious to become infectious in the individual. One skilled in the art will readily understand the meaning of an infectious microbe. The identification of infectious microbes can be performed using routine methods well known in clinical microbiology laboratories (see also Jawetz, Melnick, & Adelberg's Medical Microbiology, 25th Ed.; LANGE Basic Science (2010); Medical Microbiology, 4th ed., Samuel Baron, editor, University of Texas Medical Branch at Galveston (1996).

As used herein, the term “drug resistant” or “drug resistance” when used in reference to a microbe refers to a microbe that is resistant to the antimicrobial activity of a drug. Drug resistance is also referred to as antibiotic resistance. In some cases, a microbe that is generally susceptible to a particular antibiotic can develop resistance to the antibiotic, thereby becoming a drug resistant microbe. A “multi-drug resistant” microbe is one that is resistant to more than one drug having antimicrobial activity. One skilled in the art can readily determine if a microbe is drug resistant using routine laboratory techniques that determine the susceptibility or resistance of a microbe to a drug or antibiotic.

The invention provides a method of killing an infectious microbe. The method can include administering an effective amount of transferrin to an individual having a microbial infection, wherein the transferrin has microbicidal activity, thereby reducing survival of the infectious microbe in the individual. The infectious microbe can be, for example, a gram positive bacterium, a gram negative bacterium or a fungus. In a particular embodiment, the microbe can be a bacterium of a genus selected from Staphylococcus, Acinetobacter, Klebsiella, Enterobacter, Enterococcus, Pseudomonas, Stenotrophomonas, Escherichia, and Salmonella. In another embodiment, the microbe can be a fungus of a genus selected from Candida, Mucorales, Aspergillus, Cryptococcus, Histoplasma, Pneumocystis and Stachybotrys. It is understood that these and other genera or particular species of bacteria or fungi, as disclosed herein, can be used in a method of the invention directed to killing an infectious microbe. In still another embodiment, the infectious microbe can be drug resistant or multi-drug resistant.

The invention additionally provides a method of prophylactically treating an individual to decrease the likelihood of contracting a microbial infection. The method can include administering an effective amount of transferrin to an individual, wherein the transferrin has microbicidal activity, thereby decreasing the likelihood that the individual will contract a microbial infection. The infectious microbe can be, for example, a gram positive bacterium, a gram negative bacterium or a fungus. In a particular embodiment, the microbe can be a bacterium of a genus selected from Staphylococcus, Acinetobacter, Klebsiella, Enterobacter, Escherichia, and Salmonella. In another embodiment, the microbe can be a fungus of a genus selected from Candida, Aspergillus, Cryptococcus, Histoplasma, Pneumocystis and Stachybotrys. In still another embodiment, the infectious microbe can be drug resistant or multi-drug resistant. It is understood that these and other genera or particular species of bacteria or fungi, as disclosed herein, can be used in a method of the invention directed to prophylactically treating an individual. Thus, the methods of the invention are applicable to any pathogenic microbe, including but not limited to those described herein.

As disclosed herein, transferrin kills both gram positive bacteria, including Staphylococcus aureus, and Gram negative bacteria such as Acinetobacter baumannii, and fungi, including Candida albicans. The primary mechanism by which transferrin kills microbes is by sequestering iron so that microbes cannot access free iron for growth. In addition, transferrin can damage the cell membrane of Gram negative bacilli by sequestering divalent cations that are necessary to stabilize lipopolysaccharide complexes. The results described herein using recombinant transferrin have not previously been described, wherein transferrin is shown to be effective at killing microbes in vitro and treating infection in vivo (see Example I). The microbicidal activity of transferrin has been demonstrated to be broad, including bacteria and fungi. The fact that the treatment acts by blocking access to host iron rather than by directly acting on bacterial targets makes utilizing transferrin as unlikely to develop resistance. Additionally, transferrin is expected to be extremely safe since it is based on a normal human protein. By administering the protein at much higher concentrations than are normally found in human serum, therapeutic levels are achieved. Based on the broad spectrum activity of transferrin described herein, the fact that it is a host protein, and thus is highly likely to be safe to administer to humans, and the fact that it is already commercially available in good manufacturing practice (GMP)-grade form, transferrin is represents an alternative therapeutic agent for treating or preventing a microbial infection. Such a use is particularly advantageous for treating or preventing infection by a drug resistant microbe. Thus, transferrin can be used to treat infections of a variety of types, with a lowered potential to induce resistance since it does not act on bacterial targets and instead acts by hiding host iron from microbes.

In addition, the results disclosed herein, in which in vivo studies in mice showed that transferrin dramatically increased survival of mice systemically infected with bacteria (exemplified with S. aureus or A. baumannii) or fungi (exemplified with C. albicans) (see Example I), indicate that the methods of the invention are particularly useful for treating septicemia, a dangerous condition for which the availability of additional therapeutic options such as those described herein would be particularly beneficial. Thus, in certain embodiments, the invention provide a method for treating septicemia in an individual. Such methods can include administering an effective amount of transferrin to an individual having septicemia, thereby treating the individual. The transferrin administered to the individual can have antimicrobial activity. The infectious microbe can be, for example, a gram positive bacterium, a gram negative bacterium or a fungus. In a particular embodiment, the microbe can be a bacterium of a genus selected from Staphylococcus, Acinetobacter, Klebsiella, Enterobacter, Escherichia, and Salmonella. In another embodiment, the microbe can be a fungus of a genus selected from Candida, Aspergillus, Cryptococcus, Histoplasma, Pneumocystis and Stachybotrys. In still another embodiment, the infectious microbe can be drug resistant or multi-drug resistant. It is understood that these and other genera or particular species of bacteria or fungi, as disclosed herein, can be used in a method of the invention directed to treating septicemai. Thus, the methods of the invention are applicable to any pathogenic microbe, including but not limited to those described herein.

In recent years the critical role of iron in microbial growth and pathogenesis has garnered increasing attention. Attempts have been made to block iron uptake of microbes as a method of treating fungal and bacterial infections, and pathogens as diverse as Staphylococcus aureus, Acinetobacter baumannii, and the fungus Candida albicans, have all been shown to have substantial requirements for exogenous acquisition of iron. However, to date, no clinically viable iron-blockade option has successfully been deployed clinically, and a recent randomized, controlled trial of patients with mucormycosis suggested that small molecule-mediated iron chelation may not be as effective as previously believed. To date, no other form of iron blockade or chelation has been successfully deployed as a treatment for infections clinically, even though it is known that virtually all microbes require access to iron in order to be able to grow.

As discussed above, transferrin was previously known to have activity against gram negative bacteria. However, the results described herein are unexpected in that it was not previously recognized that transferrin has microbicidal activity or that transferrin would be effective as an in vivo therapy (see Example I). For example, transferrin was previously shown to have bacteriostatic activity against Bacillus anthracis (Rooijakkers et al., J. Biol. Chem. 285:27609-27613 (2010)). However, contrary to the present studies, Rooijakkers et al. indicated that transferrin had no growth inhibitory activity against Staphylococcus aureus or Streptococcus pneumoniae. Therefore, the results described herein that transferrin has microbicidal activity and is effective in vivo against a broad range of organisms, including gram positive and gram negative bacteria and fungi, are unexpected. Thus the methods of the invention can be applied to a wide range of microbes, including but not limited to those describe herein. Since the microbicidal and in vivo effectiveness of transferrin was not previously recognized, the methods therefore provide unexpected results. Nevertheless, in a particular embodiment, the invention provides a method of killing an infectious microbe or prophylactically treating an individual to decrease the likelihood of contracting a microbial infection, with the proviso that Bacillus anthracis and/or Bacillus species are excluded as the infectious microbe.

The methods of the invention have a variety of uses. For example, the uses can include preventing (prophylaxis) of infection in high risk settings. For example, patients that are immunocompromised due to drug treatment, such as transplant patients, neutropenic patients, burn patients, cancer patients, or due to disease, such as patients with cancer, immune-system disorders, AIDS, cystic fibrosis, or any condition or disease that reduces immunocompetency, and the like, can be treated prophylactically with transferrin to decrease the likelihood of contracting a microbial infection. It may also be possible to modify transferrin to make it better able to sequester iron, and better able to kill microbes.

As described above, an infectious microbe can be a pathogenic bacterium or fungus. Exemplary genera of pathogenic bacteria include, but are not limited to, Acinetobacter, Bacillus, Bordetella, Borrelia, Brucella, Campylobacter, Chlamydia, Chlamydophila, Clostridium, Corynebacterium, Enterobacter, Enterococcus, Escherichia, Francisella, Haemophilus, Helicobacter, Klebsiella, Legionella, Leptospira, Listeria, Mycobacterium, Mycoplasma, Neisseria, Pseudomonas, Rickettsia, Salmonella, Shigella, Staphylococcus, Streptococcus, Stenotrophomonas, Treponema, Vibrio, Yersinia, and the like. Exemplary pathogenic species include, but are not limited to, Acinetobacter baumanii, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterobacter sazakii, Enterobacter agglomerans, Enterobacter cloacae, Enterobacter aerogenes, Enterococcus faecalis, Enterococcus faecium, Escherichia coli, Francisella tularensis, Haemophilus influenzae, Helicobacter pylori, Klebsiella pneumoniae, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Salmonella enterica, Shigella sonnei, Staphylococcus aureus, Staphylococcus epidermidis, Staphylococcus saprophyticus, Streptococcus agalactiae, Streptococcus pneumoniae, Streptococcus pyogenes, Stenotrophomonas maltophilia, Treponema pallidum, Vibrio cholerae, Yersinia pestis, and the like.

Exemplary diseases or conditions caused by infectious bacteria include, but are not limited to, nosocomial pneumonia, infections associated with continuous ambulatory peritoneal dialysis (CAPD), or catheter-associated bacteruria (Acinetobacter baumanii); cutaneous anthrax, pulmonary anthrax, and gastrointestinal anthrax (Bacillus anthracis); whooping cough and secondary bacterial pneumonia (Bordetella pertussis); Lyme disease (Borrelia burgdorferi); brucellosis (Brucella abortus, Brucella canis, Brucella melitensis, Brucella suis); acute enteritis (Campylobacter jejuni); community-acquired respiratory infection (Chlamydia pneumoniae); nongonococcal urethritis (NGU), lymphogranuloma venereum (LGV), trachoma, conjunctivitis of the newborn (Chlamydia trachomatis); psittacosis (Chlamydophila psittaci); botulism (Clostridium botulinum); pseudomembranous colitis (Clostridium difficile); gas gangrene, acute food poisoning, anaerobic cellulitis (Clostridium perfringens); tetanus (Clostridium tetani); diphtheria (Corynebacterium diphtheriae); bacteremia, lower respiratory tract infections, skin and soft-tissue infections, urinary tract infections (UTIs), endocarditis, intra-abdominal infections, septic arthritis, osteomyelitis, ophthalmic infections, wound infections, nosocomial infections (Enterobacter cloacae, Enterobacter aerogenes); skin, respiratory, and urinary infections (Enterococcus cloacae); nosocomial infections (Enterococcus faecalis, Enterococcus faecium); urinary tract infections (UTI), diarrhea, meningitis in infants, hemorrhagic colitis, hemolytic-uremic syndrome (Escherichia coli); tularemia (Francisella tularensis); bacterial meningitis, upper respiratory tract infections, pneumonia, bronchitis (Haemophilus influenzae); peptic ulcer (Helicobacter pylori); pneumonia, infections of the urinary tract, biliary tract and surgical wounds (Klebsiella pneumoniae); Legionnaire's disease, Pontiac fever (Legionella pneumophila); leptospirosis (Leptospira interrogans); listeriosis (Listeria monocytogenes); leprosy (Hansen's disease) (Mycobacterium leprae); tuberculosis (Mycobacterium tuberculosis); mycoplasma pneumonia (Mycoplasma pneumoniae); skin lesions and ulcers (Mycobacterium ulcerans); gonorrhea, ophthalmia neonatorum, septic arthritis (Neisseria gonorrhoeae); meningococcal disease including meningitis, Waterhouse-Friderichsen syndrome (Neisseria meningitidis); pseudomonas infection (localized to eye, ear, skin, urinary, respiratory or gastrointestinal tract or CNS, or systemic with bacteremia, secondary pneumonia bone and joint infections, endocarditis, skin, soft tissue or CNS infections) (Pseudomonas aeruginosa); rocky mountain spotted fever (Rickettsia rickettsii); typhoid fever type salmonellosis (Salmonella typhi); salmonellosis with gastroenteritis and enterocolitis (Salmonella typhimurium); bacillary dysentery/shigellosis (Shigella sonnei); coagulase-positive staphylococcal infections, including localized skin infections, diffuse skin infection (Impetigo), deep, localized infections, acute infective endocarditis, septicemia, necrotizing pneumonia, toxinoses such as toxic shock syndrome and staphylococcal food poisoning (Staphylococcus aureus); infections of implanted prostheses, e.g. heart valves and catheters (Staphylococcus epidermidis); Ccystitis in women (Staphylococcus saprophyticus); meningitis and septicemia in neonates, endometritis in postpartum women, opportunistic infections with septicemia and pneumonia (Streptococcus agalactiae); acute bacterial pneumonia and meningitis in adults, otitis media and sinusitis in children (Streptococcus pneumoniae); streptococcal pharyngitis, scarlet fever, rheumatic fever, impetigo and erysipelas puerperal fever, necrotizing fasciitis (Streptococcus pyogenes); pulmonary infections, colonization of prosthetic material such as catheters or endotracheal or tracheostomy tubes, pneumonia, urinary tract infection, bacteremia, soft tissue infection, ocular infection, endocarditis, meningitis (Stenotrophomonas maltophilia); syphilis (Treponema pallidum); cholera (Vibrio cholerae); plague, including bubonic plague and pneumonic plague (Yersinia pestis), and the like. Exemplary drug resistant bacteria include, but are not limited to, Enterococcus faecium, Staphylococcus aureus, Klebsiella pneumoniae, Acinetobacter baumanii, Pseudomonas aeruginosa and Enterobacter spp.

Fungal infection, or mycoses, of humans and animals include, for example, superficial fungal infections that affect the outer layers of skin; fungal infections of the mucous membranes including the mouth (thrush), vaginal and anal regions, such as those caused by Candida albicans, and fungal infections that affect the deeper layers of skin and internal organs are capable of causing serious, often fatal illness. Fungal infections are well known in the art and include, for example, mucormycosis, entomophthoromycosis, aspergillosis, cryptococcosis, candidiasis, histoplasmosis, coccidiomycosis, paracoccidiomycosis, fusariosis (hyalohyphomycoses), blastomycosis, penicilliosis or sporotrichosis. These and other fungal infections can be found described in, for example, Merck Manual, Sixteenth Edition, 1992, and in Spellberg et al., Clin. Microbio. Rev. 18:556-69 (2005). The exemplary fungal conditions described above are described further below. Exemplaryn genera of pathogenic fungi include, but are not limited to, Candida, Aspergillus, Cryptococcus, Histoplasma, Pneumocystis and Stachybotry.

As used herein, the term “entomophthoromycosis” is intended to mean a fungal condition caused by fungi of the subphylum Entomophthomycotina. The Entomophthoromycoses are causes of subcutaneous and mucocutaneous infections known as entomophthoromycosis, which largely afflict immunocompetent hosts in developing countries.

As used herein, the term “mucormycosis” is intended to mean a fungal condition caused by fungi of the subphylym Mucormycotina, order Mucorales. Mucormycosis is a life-threatening fungal infection almost uniformly affecting immunocompromised hosts in either developing or industrialized countries. Fungi belonging to the order Mucorales are distributed into six families, all of which can cause cutaneous and deep infections. Species belonging to the family Mucoraceae are isolated more frequently from patients with mucormycosis than any other family. Among the Mucoraceae, Rhizopus oryzae (Rhizopus arrhizus) is a common cause of infection. Other exemplary species of the Mucoraceae family that cause a similar spectrum of infections include, for example, Rhizopus microsporus var. rhizopodiformis, Absidia corymbifera, Apophysomyces elegans, Mucor species, Rhizomucor pusillus and Cunninghamella spp (Cunninghamellaceae family). Mucormycosis is well known in the art and includes, for example, rinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis.

It is understood by those skilled in the art that “entomophthoromycosis” and “mucormycosis” were previously considered to overlap with zygomycosis but that taxonomic changes within the fungi has resulted in changes in nomenclature (see Kwon-Chung, Clin. Infect. Dis. 54:S8-15 (2012)). It is further understood by those skilled in the art that conditions previously considered to be zygomycoses are included within “entomophthoromycosis” and “mucormycosis” as understood by those skilled in the art under current taxonomy. Therefore, the invention also relates to conditions that would have previously been classified as zygomycosis, now classified as “entomophthoromycosis” or “mucormycosis” as discussed above.

As used herein, the term “candidiasis” is intended to mean a fungal condition caused by fungi of the genus Candida. Candidiasis can occur in the skin and mucous membranes of the mouth, respiratory tract and/or vagina as well as invade the bloodstream, especially in immunocompromised individuals. Candidiasis also is known in the art as candidosis or moniliasis. Exemplary species of the genus Candida include, for example, Candida albicans, Candida krusei, Candida tropicalis, Candida glabrata and Candida parapsilosis.

As used herein, the term “aspergillosis” is intended to mean the group of diseases caused by the genus Aspergillus. The symptoms include, for example, fever, cough, chest pain and/or breathlessness. Patients with a weakened immune systems or who suffer from a lung condition are particularly susceptible to aspergillosis. Exemplary forms of this fungal condition include allergic aspergillosis, which affects asthma, cystic fibrosis and sinusitis patients); acute invasive aspergillosis, which shows increased incidence in patients with weakened immunity such as in cancer patients, patients undergoing chemotherapy and AIDS patients; disseminated invasive aspergillosis, which is widespread throughout the body, and opportunistic Aspergillus infection, which is characterized by inflammation and lesions of the ear and other organs. Aspergillus is a genus of around 200 fungi. Aspergillus species causing invasive disease include, for example, Aspergillus fumigatus and Aspergillus flavus. Aspergillus species causing allergic disease include, for example, Aspergillus fumigatus and Aspergillus clavatus. Other exemplary Aspergillus infectious species include, for example, Aspergillus terreus and Aspergillus nidulans.

As used herein, the term “cryptococcosis” is intended to mean a fungal condition caused by the genus Cryptococcus. Cryptococcosis, also known as Busse-Buschke disease, generally manifests as a systemic infection that can affect any organ of the body including, for example, the lungs, skin, or other body organs, but most often occurs in the central nervous system such as the brain and meninges. Cryptococcosis is an opportunistic infection for AIDS, although patients with Hodgkin's or other lymphomas or sarcoidosis or those receiving long-term corticosteroid therapy are also at increased risk. Symptoms include, for example, chest pain, dry cough, swelling of abdomen, headache, blurred vision and confusion. Exemplary forms of this fungal condition include cutaneous cryptococcosis such as those occurring in wounds, pulmonary cryptococcosis and Cryptococcal meningitis. Cryptococcal meningitis can result from dissemination of Cryptococcus neoformans from either an observed or unappreciated pulmonary infection generally in immunocompromised patients. C. gattii generally causes infections in immunocompetent people. Detection of cryptococcal antigen (capsular material) by culture of CSF, sputum and urine provides one useful method of diagnosis. Blood cultures also can be positive in heavy infections.

As used herein, the term “histoplasmosis” is intended to mean a fungal condition caused by the genus Histoplasma, including the infectious disease caused by the inhalation of spores of Histoplasma capsulatum. Histoplasmosis also is known in the art as Darling's disease. The condition can be asymptomatic, but also can progress to acute pneumonia or an influenza like illness, primarily affects the lungs. Histoplasmosis also can spread to other organs and systems in the body. As with other disseminated forms of fungal conditions, this disseminated histoplasmosis can be fatal. Symptoms can occur within 3 to 17 days after exposure. However, in undisseminated forms, it can be common for infected individuals to exhibit no apparent ill effects. The acute respiratory disease can be characterized by respiratory symptoms, a general ill feeling, fever, chest pains, and a dry or nonproductive cough. Distinct patterns also can be seen on a chest x-ray. Chronic lung disease resembles tuberculosis and can worsen over months or years.

As used herein, the term “coccidiomycosis” is intended to mean a fungal condition caused by the genus Coccidioides. Included in the meaning of the term is the infectious respiratory disease caused by Coccidioides immitis or C. posadasii, particularly through inhalation of spores, and which is characterized by fever and various respiratory symptoms. Coccidiomycosis also is known in the art as coccidioidomycosis and valley fever. Systemic coccidiomycosis can spread from the respiratory tract to, for example, the skin, bones, and central nervous system. Manifestations of the condition range from complete absence of symptoms to systemic infection and death. For example, symptomatic infection (about 40% of cases) can present as an influenza-like illness with fever, cough, headaches, rash, and myalgia (muscle pain). Some patients can fail to recover and develop chronic pulmonary infection or widespread disseminated infection (affecting meninges, soft tissues, joints, and bone). Severe pulmonary disease can develop in, for example, HIV-infected and other immunocompromised persons.

As used herein, the term “paracoccidiomycosis” is intended to mean a fungal condition caused by the genus Paracoccidioides including, for example, a chronic mycosis caused by Paracoccidioides brasiliensis. Paracoccidiomycosis is characterized by primary lesions of the lungs with dissemination to many internal organs, by conspicuous ulcerative granulomas of the mucous membranes of the cheeks and nose with extensions to the skin, and by generalized lymphangitis. Paracoccidiomycosis also is known in art as paracoccidioidomycosis, Almeida's disease, Lutz-Splendore-Almeida disease, paracoccidioidal granuloma and South American blastomycosis.

As used herein, the term “fusariosis” or ““hyalohyphomycoses” is intended to mean a fungal condition caused by the genus fusarium. Fusarium species causing the condition include, for example, F. solani, F. oxysporum and F. moniliforme. Infections include keratitis, onychomycosis and occasionally peritonitis and cellulitis. Risk factors for disseminated fusariosis include severe immunosuppression (neutropenia, lymphopenia, graft-versus-host disease, corticosteroids), colonisation and tissue damage. Among immunocompetent patients, tissue breakdown (as caused by trauma, severe burns or foreign body) is the risk factor for fusariosis. Clinical presentation includes refractory fever, skin lesions and sino-pulmonary infections. Skin lesions can lead to diagnosis in many patients and precede fungemia by approximately 5 days. Disseminated fusariosis can be diagnosed by, for example, blood cultures and other well known methods described above and below.

As used herein, the term “blastomycosis” is intended to mean a fungal condition caused by the genus blastomycete, generally originating as a respiratory infection, and usually spreading to the lungs, bones, and skin. Blastomycosis is characterized by multiple inflammatory lesions of the skin, mucous membranes, or internal organs. Blastomyces dermatitidis is one species prevalent causative species. Symptoms of blastomycosis include, for example, a flulike illness with fever, chills, myalgia, headache, and a nonproductive cough; an acute illness resembling bacterial pneumonia, with symptoms of high fever, chills, a productive cough, and pleuritic chest pain; a chronic illness that mimics tuberculosis or lung cancer, with symptoms of low-grade fever, a productive cough, night sweats, and weight loss; a fast, progressive, and severe disease that manifests as ARDS, with fever, shortness of breath, tachypnea, hypoxemia, and diffuse pulmonary infiltrates; skin lesions; bone lytic lesions; prostatitis, and/or laryngeal involvement causing hoarseness.

As used herein, the term “penicilliosis” is intended to mean a fungal condition caused by the genus penicillium. An exemplary species is penicillium marneffei, which is a prevalent cause of opportunistic fungal infections in immunocompromised individuals. Diagnosis is can be made by identification of the fungi from clinical specimens. Biopsies of skin lesions, lymph nodes, and bone marrow can demonstrate the presence of organisms on histopathology. Symptoms include, for example, fever, skin lesions, anemia, generalized lymphadenopathy, and hepatomegaly.

As used herein, the term “sporotrichosis” is intended to mean a fungal condition caused by the genus Sporothrix, including the species S. schenckii. The condition manifests as a chronic infectious characterized by nodules or ulcers in the lymph nodes and skin.

Sporotrichosis infection can spread through the blood to other areas including, for example, infection of the joints, lungs, eye, and the genitourinary and central nervous system. Generally, disseminated sporotrichosis occurs in immunocompromised individuals such as patients with AIDS, cancer, patients undergoing chemotherapy, and transplant recipients on immunosuppressive therapy.

As used herein, the terms “effective amount” or “therapeutically effective amount” are intended to mean an amount of transferrin, or other compound, to effect a decrease in the extent, amount or rate of infection when administered to an individual. Therefore, an effective amount when used in reference to transferrin is intended to mean an amount of transferrin sufficient to ameliorate at least one symptom associated with a microbial infection, including but not limited to measuring the amount of infectious agent after treatment with transferrin.

The dosage of transferrin required to be therapeutically effective will depend, for example, on the microbial infection to be treated or prevented as well as the weight and condition of the individual, and previous or concurrent therapies. The appropriate amount considered to be an effective dose for a particular application of the method can be determined by those skilled in the art, using the guidance provided herein. For example, the amount can be extrapolated from in vitro or in vivo assays as described below. One skilled in the art will recognize that the condition of the patient needs to be monitored throughout the course of therapy and that the amount of the composition that is administered can be adjusted according to the response of the therapy.

In addition, it is understood by those skilled in the art that combination therapy can be used with transferrin. Although the use of transferrin is particularly useful with respect to treating a drug resistant microbe, it is understood that transferrin can be combined with other antibiotics, including antibacterial or antifungal agents, as desired.

The amount of transferrin included in a composition for use in methods of the invention can vary but will generally be a therapeutically effective amount or an amount that can be reconstituted or diluted to a therapeutically effective amount. Tansferrin also can be formulated in a composition in amounts greater than a therapeutically effective amount for either short or long-term storage and the end user can dilute the formulation prior to use to a desired therapeutically effective amount. The formulations containing an effective amount of constituents can contain transferrin, or other agents such as other antibiotics, if desired, alone or together with any desired excipients, surfactants, tonicifiers, salts or buffers. Dilution or reconstitution can be performed in a pharmaceutically acceptable medium that adjusts the formulation to the desired therapeutically effective amount of transferrin and includes any includes any additional excipients, surfactants, tonicifiers, salts or buffers. Pharmaceutical formulations are well known and practiced in the pharmaceutical. Any such well known formulations and pharmaceutical formulation components are applicable for use with a composition of the invention. Pharmaceutical formulations, excipients, their use, formulation and characteristics are well known in the art and can be found described in, for example, Remington: The Science and Practice of Pharmacy, supra; Williams et al., Foye's Principles of Medicinal Chemistry, 5th Ed., Lippincott Williams & Wilkins (2002); Allen et al., Ansels Pharmaceutical Dosage Forms and Drug Delivery Systems, 8th Ed., Lippincott Williams & Wilkins (2004). Similarly, surfactant, their use, formulation and characteristics are well known in the art and can be found described in, for example, Holmberg et al., Surfactants and Polymers in Aqueous Solution, supra; Surfactants: A Practical Handbook, K. Robert Lange, ed., supra, and Vogel, A. I., Vogel's Textbook of Practical Organic Chemistry, 5th ed., Pearson Education Limited (1989).

It is understood that modifications which do not substantially affect the activity of the various embodiments of this invention are also provided within the definition of the invention provided herein. Accordingly, the following examples are intended to illustrate but not limit the present invention.

### EXAMPLE I

### Treating Bacterial and Fungal Infections with Transferrin

This example describes the determination that transferrin has microbicidal and micobistatic activity against a broad spectrum of pathogens.

The ongoing crisis of antibiotic resistance demands new methods to prevent and treat infections caused by highly resistant organisms. While new antibiotics are critically needed, ultimately resistance will generally develop to any new antibiotic. Thus, policy advocates and international experts have called for increased exploration of novel strategies to treat infected patients that act to stimulate host defenses or by other mechanisms that are less likely to induce resistance. While transferrin has long been known to sequester iron in serum, recombinant transferrin has not previously been shown to be effective at killing microbes in vitro and treating infection in vivo.

As described below, it has been discovered that conditioned medium from murine embryonic stem cell cultures possessed microbicidal properties. Systematic investigation revealed that the active agent in the conditioned medium that was responsible for these properties was transferrin that was expressed and secreted by murine embryonic stem cells. Recombinant transferrin had microbicidal activity against a broad spectrum of pathogens. Based on the broad spectrum activity of transferrin, the fact that it is a host protein and therefore expected to be safe to administer to humans, and the fact that it is already commercially available in good manufacturing practice (GMP)-compliant form, transferrin is a therapeutic agent useful for treating pathogenic infections.

Materials and Methods: Stem cells. Mouse embryonic stem cells (MES) (2×10⁴) were cultured in MES media in 6 well plates. Conditioned media were collected on day 5 for further studies on antimicrobial activity.

Microbes. Candida albicans SC5314 is a clinical bloodstream isolate that is highly virulent in murine models of infection. Acinetobacter baumannii HUMC1 is a carbapenem-resistant clinical bloodstream isolate that is highly virulent in mice. Staphylococcus aureus LAC is a methicillin resistant, USA300 clinical isolate that is also virulent in mice.

Transferrin analysis. Secreted levels of transferrin were quantified by ELISA using standard methods. Transferrin was knockdown by transferrin target siRNA. Transferrin mRNA level expressions were measured by RT-PCR.

Mice. Balb/C and C3H/FeJ mice were used.

Initial studies were carried on on pluripotent stem cells to identify factors produced by the stem cells. Mouse embryonic stem (MES) cells and induced pluripotent stem (iPS) cells conditioned media were analyzed by HPLC and MALDI-TOF analysis to identify factors present in conditioned but not control media. Transferrin mRNA level expressions were detected by RT-PCR. Secreted levels of transferrin were quantified by ELISA and functionality was assayed for by flow cytometry using two different fluorochromes (CD71 and SSEA-1). A proliferation assay kit was used to detect stem cell proliferation.

Conditioned media from both MES and iPS cells contained an HPLC fraction that was not present in unconditioned (control) media. By MALD-TOF analysis the predominant material present in the conditioned fraction was transferrin. Quantitative ELISA confirmed that the concentration of mouse transferrin increased daily in media conditioned by stem cells, such that levels at day 5 were 50 to 100-fold greater than on day 0. Transferrin enhanced the stem cell proliferation. These results showed that mouse pluripotent stem cells, including both MES and iPS, secrete functional transferrin during growth. The transferrin supports enhanced stem cell replication, and blockade of transferrin reduces stem cell replication.

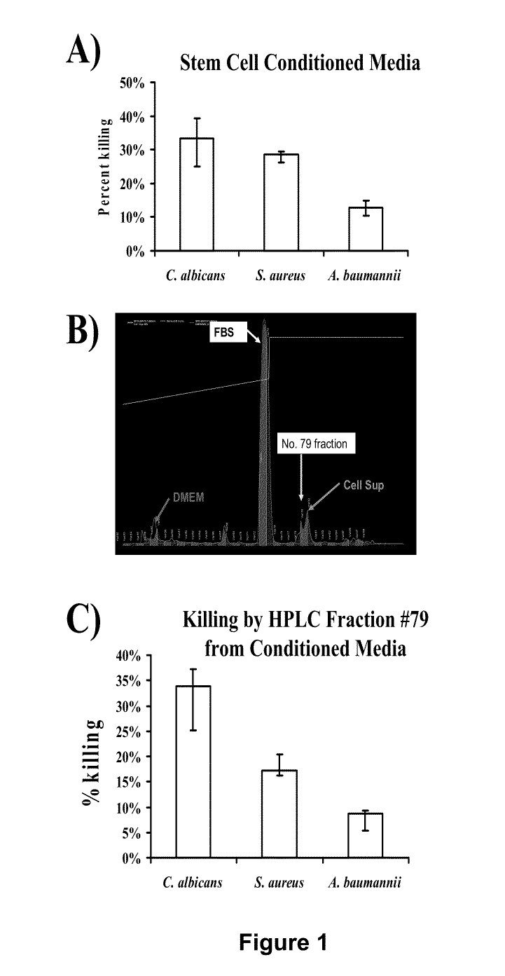

Stem cell conditioned media were found to have antimicrobial activity. As shown in FIG. 1, a biochemical fraction in mouse embryonic stem (MES) cell conditioned media had broad antimicrobial effects. Conditioned media from stem cells was collected at day 5. As shown in FIG. 1A, cell culture media harvested after 5 days of MES culture resulted in the killing of bacteria or fungi after 1 hour in the conditioned cell culture media. Representative microbes were utilized for gram positive bacteria (Staphylococcus aureus), gram negative bacteria (Acinetobacter baumannii), and fungi (Candida albicans).

HPLC was used to identify fractions that were distinct between conditioned and control culture media (DMEM). As shown in FIG. 1B, fraction 79 contained a peak distinct from control medium. The microbicidal activity of HPLC fraction 79 was confirmed. As shown in FIG. 1C, HPLC-purified fraction 79 recapitulated conditioned media microbicidal effects.

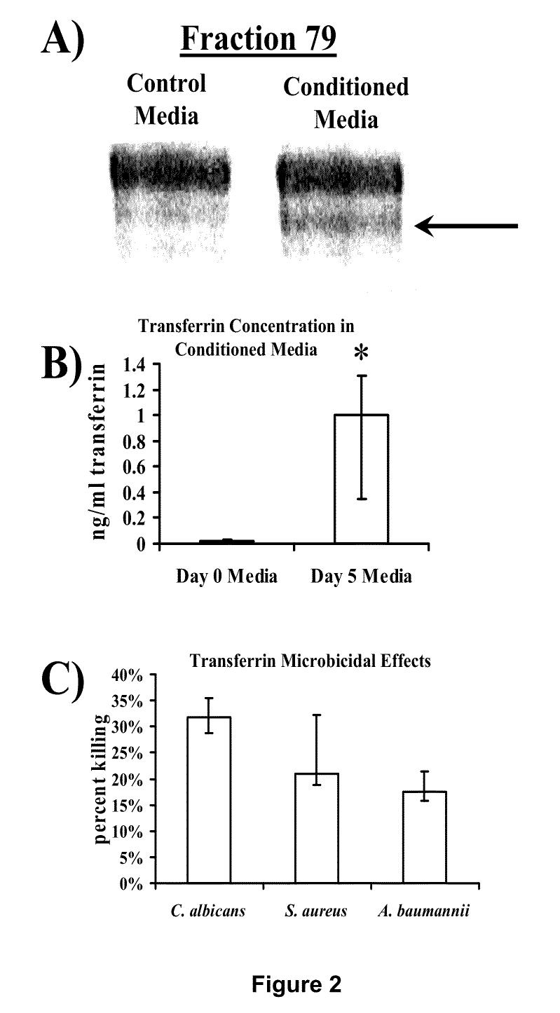

HPLC fraction 79 from conditioned media was further characterized. HPLC fraction 79 was analyzed by polyacrylamide gel electrophoresis (PAGE). As shown in FIG. 2, a silver-stained PAGE gel of Fraction 79 revealed a single band distinguishing conditioned from control media. Fraction 79 was also characterized by MALDI-TOF and determined to contain transferrin. ELISA analysis was also performed, and FIGS. 2B shows that ELISA confirmed the presence of higher quantities of transferrin in conditioned media than control media. To confirm that transferrin was the component in fraction 79 having microbicidal activity, recombinant transferrin was tested for antimicrobial activity on C. albicans, S. aureus, and A. baumannii. As shown in FIG. 2C, recombinant transferrin also exhibited microbial killing activity. These results demonstrate that transferrin has broad antimicrobial effects, in particular microbicidal activity.

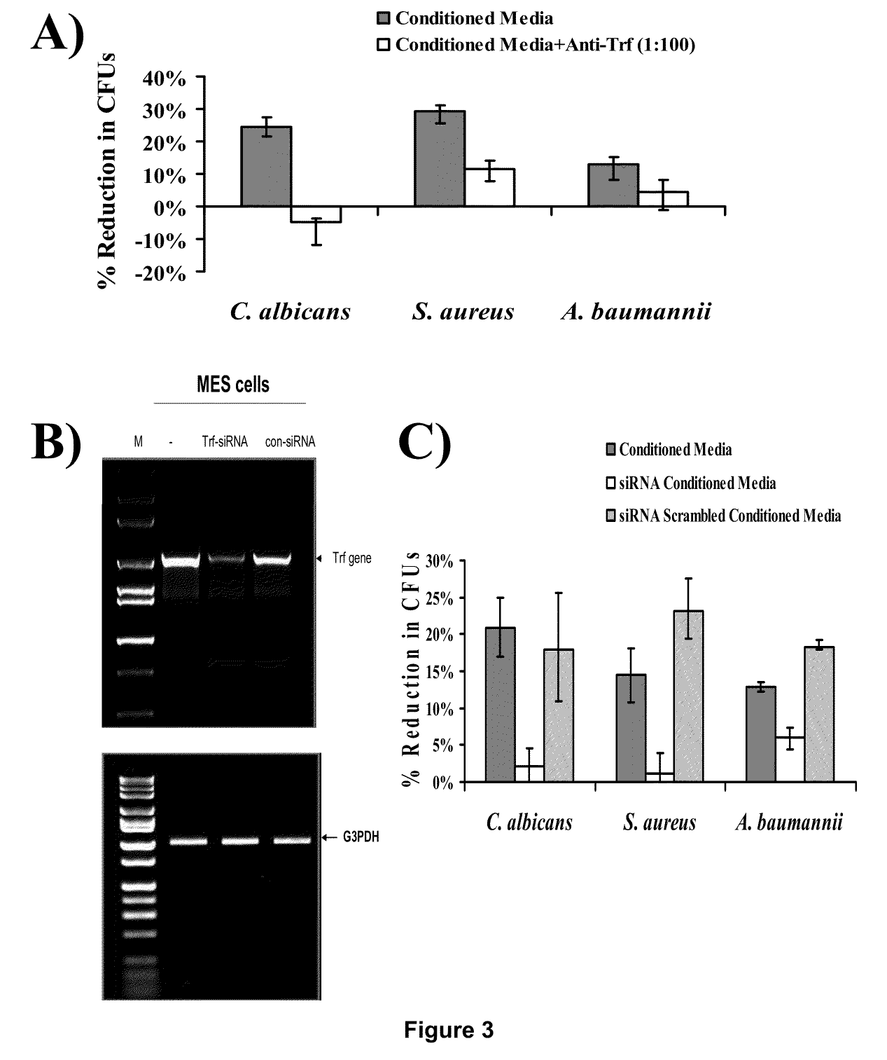

Further experiments were performed to confirm that transferrin was the component in conditioned media responsible for the microbicidal activity. Conditioned media alone or with the inclusion of anti-transferrin antibody was tested for killing activity on C. albicans, S. aureus and A. baumannii. As shown in FIG. 3A, the killing activity of the conditioned medium was reduced in the presence of anti-transferrin antibody. Experiments were also performed using siRNA targeted to transferrin to reduce transferrin expression in MES cells. As shown in FIG. 3B, siRNA targeted to transferrin reduced transferrin expression, whereas control siRNA did not. Glyceraldehyde-3-phosphate dehydrogenase (G3PDH) was measured to confirm loading of an equivalent number of cells in each lane. As shown in FIG. 3C, after transfection of stem cells with anti-transferrin siRNA constructs that suppressed Trf gene expression, the microbicidal effects were blocked. These results confirm that transferrin mediated the microbicidal effects of stem cell conditioned media.

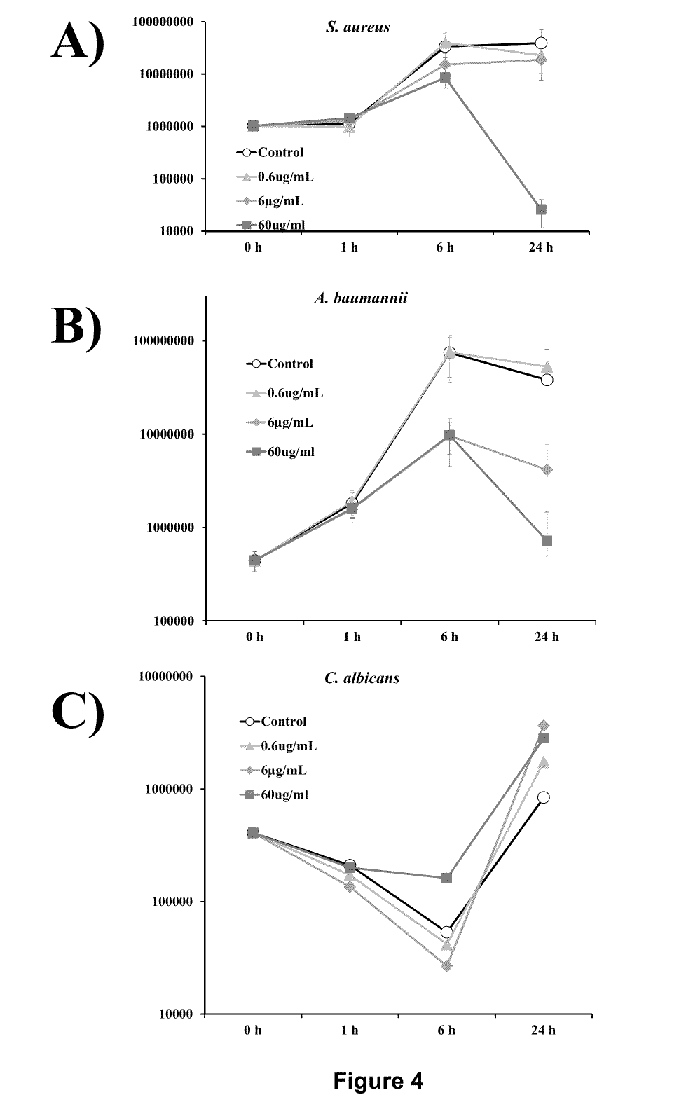

In order to further assess the antimicrobial activity of transferrin, time-kill analyses were conducted on bacterial and fungal organisms. The bacteria S. aureus and A. baumannii, and the fungus C. albicans were inoculated and grown in the presence of rhTransferrin at varying concentrations (0.6 μg/mL, 6 μg/mL or 60 μg/mL) or grown in the absence of rhTransferrin (control). The cultures were serially sampled and the organism densities in the cultures were measured and compared. The results show that transferrin is microbistatic, not microbicidal, under these in vitro culture conditions (FIGS. 4A-C). Nevertheless, treatment with transferrin does substantially prevent bacterial growth at both 6 hrs and 24 hrs time points (FIGS. 4A and 4B) and can prevent fungal growth in a dose dependent manor at 6 hrs, which can subside by 24 hrs (FIG. 4C).

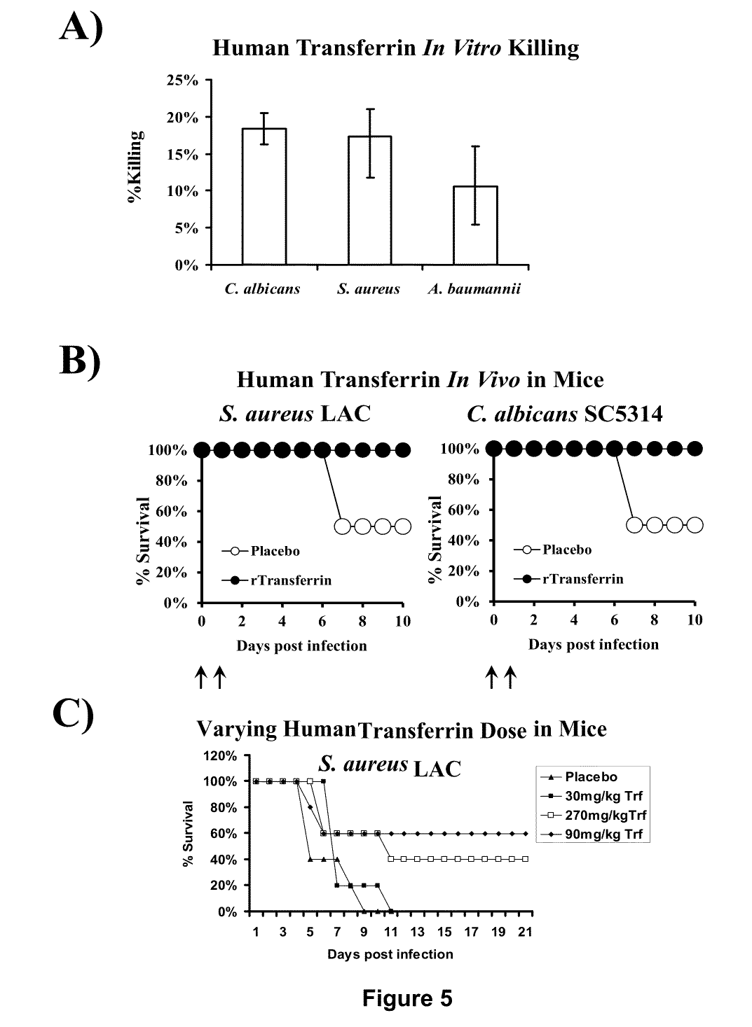

The antimicrobial activity of transferrin was also tested in Balb/c mice. To confirm that human transferrin, in addition to mouse transferrin as described above, was active, kill assays were repeated using human recombinant transferrin. As shown in FIG. 5A, recombinant human transferrin also exhibited microbicidal activity against C. albicans, S. aureus and A. baumannii. Human recombinant transferrin was also tested in vivo. Briefly, mice were infected intravenously via the tail-vein with S. aureus or C. albicans in the presence of placebo or transferrin, and mouse survival was measured. As shown in FIG. 5B, human recombinant transferrin administered to mice (n=2 per group) in a small, preliminary pilot study confirmed that 3 mg/kg/d could be delivered safely. In addition, FIG. 4B shows that mice receiving transferrin had 100% survival, whereas mice receiving placebo had about 50% survival.

Human transferrin was also tested at various doses for its effect on survival of S. aureus infected Balb/c mice. Mice (n=5 per group) were treated with 30, 90 and 270 mg/kg intravenously (iv) of rhTransferrin once daily for total 3 days. As shown in FIG. 4C, administration of the lowest dose of 30 mg/kg extended the days of survival of the mice relative to placebo, although ultimately all mice died. At the higher doses of 90 and 270 mg/kg, there was a significantly higher survival rate, reaching a plateau at about 60% and 40%, respectively, for the two doses. The survival of the mice reaching the plateau continued for up to 21 days post-infection, indicating that the mice appeared to likely clear the infection. These results indicate that transferrin is an effective antimicrobial agent in vivo.

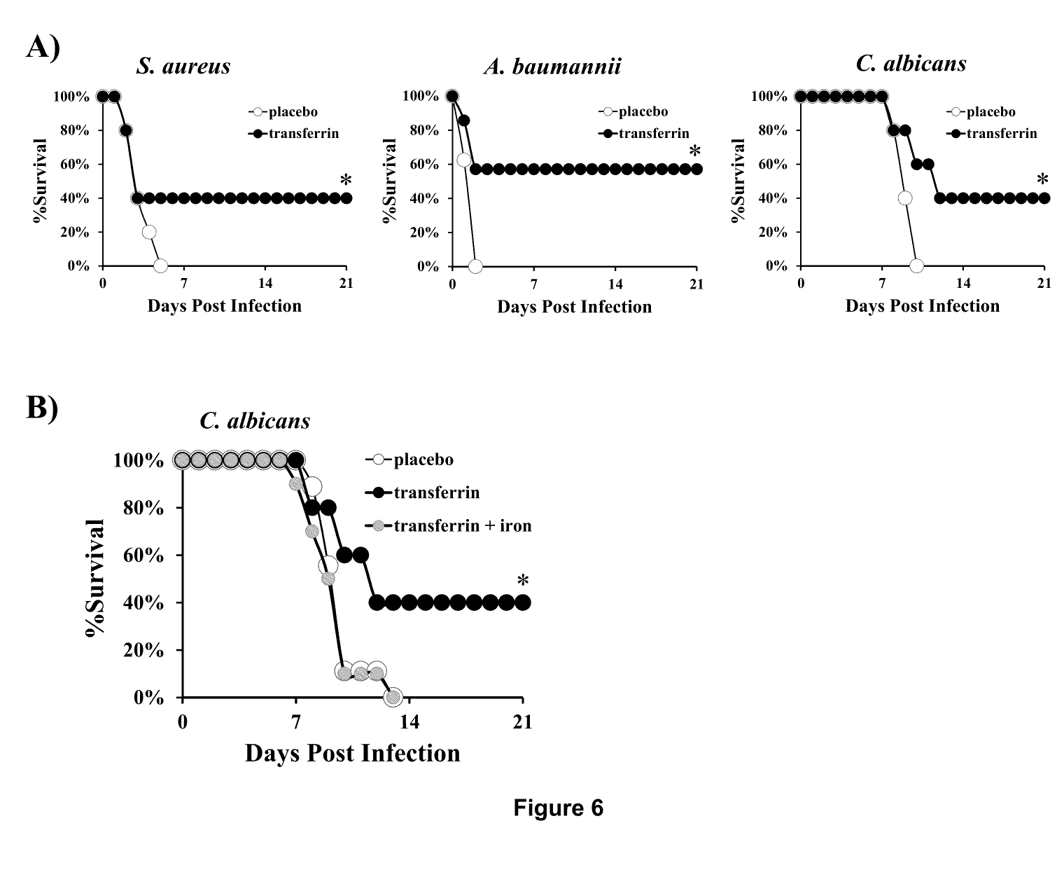

Human transferrin was still further tested for its effect on survival of S. aureus, A. baumannii or C. albicans infected mice. Balb/c mice (n=10 per group) were infected with 5×10⁷ S. aureus LAC or 1×10⁵ C. albicans SC5314, whereas C3H/FEJ mice (n=10 per group) were infected with 2×10⁷ A. baumannii HUMC1. The mice were treated intravenously (iv) with 90 mg/kg of rhTransferrin once daily for 4 days total. As shown in FIG. 6A, administration of the human transferrin significantly improved the survival of the mice relative to placebo for 21 days post infection. All surviving mice appeared clinically well. These results further show that transferrin is an effective antimicrobial agent in vivo.

In order to identify the mechanism of human transferrin's antimicrobial activity, the effect of surplus free iron on mice survival was also assayed. Balb/c mice (n=10 per group) were infected with 1×10⁵ C. albicans SC5314 and treated intravenously (iv) with either rhTransferrin alone (90 mg/kg/d×4 d) or rhTransferrin and FeCl₃ (0.4 mg/kb). The addition of free iron saturates the transferrin in the serum, thereby maintaining some serum iron in the mice. Mice treated with the rhTransferrin and FeCl₃ survived the same amount of time as the placebo treated mice (FIG. 6C). Thus, without being bound by theory, the antimicrobial activity of transferrin is likely attributed to its iron sequestering activity so that microbes cannot access free iron for growth.

These results indicate that recombinant transferrin has microbicidal and microbistatic activity against a broad spectrum of pathogens. Administration of exogenous human transferrin significantly improved survival of mice infected with either S. aureus, A. baumannii and C. albicans, indicating that transferrin is an effective antimicrobial agent in vivo.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method of killing an infectious microbe, comprising administering an effective amount of transferrin to an individual having a microbial infection, wherein said transferrin has microbicidal activity, thereby reducing survival of the infectious microbe in the individual.

2. The method of claim 1, wherein the infectious microbe is selected from a gram positive bacterium, a gram negative bacterium and a fungus.

3. The method of claim 1, wherein the infectious microbe is a bacterium of a genus selected from Staphylococcus, Acinetobacter, Klebsiella, Enterobacter, Enterococcus, Pseudomonas, Stenotrophomonas, Escherichia, and Salmonella.

4. The method of claim 1, wherein the infectious microbe is a fungus of a genus selected from Candida, Mucorales, Aspergillus, Cryptococcus, Histoplasma, and Pneumocystis.

5. The method of claim 1, wherein the infectious microbe is drug resistant.

6. The method of claim 5, wherein the infectious microbe is multi-drug resistant.

7. A method of prophylactically treating an individual to decrease the likelihood of contracting a microbial infection, comprising administering an effective amount of transferrin to an individual, wherein said transferrin has microbicidal activity, thereby decreasing the likelihood that the individual will contract a microbial infection.

8. The method of claim 7, wherein the infectious microbe is selected from a gram positive bacterium, a gram negative bacterium or a fungus.

9. The method of claim 7, wherein the infectious microbe is a bacterium of a genus selected from Staphylococcus, Acinetobacter, Klebsiella, Enterobacter, Enterococcus, Pseudomonas, Stenotrophomonas, Escherichia, and Salmonella.

10. The method of claim 7, wherein the infectious microbe is a fungus of a genus selected from Candida, Mucorales, Aspergillus, Cryptococcus, Histoplasma, and Pneumocystis.

11. The method of claim 7, wherein the infectious microbe is drug resistant.

12. The method of claim 11, wherein the infectious microbe is multi-drug resistant.

13. A method for treating septicemia in an individual comprising administering an effective amount of transferrin to an individual having septicemia, thereby treating the individual.

14. The method of claim 13, wherein said septicemia is caused by an infectious microbe selected from a gram positive bacterium, a gram negative bacterium or a fungus.

15. The method of claim 14, wherein the infectious microbe is a bacterium of a genus selected from Staphylococcus, Acinetobacter, Klebsiella, Enterobacter, Enterococcus, Pseudomonas, Stenotrophomonas, Escherichia, and Salmonella.

16. The method of claim 14, wherein the infectious microbe is a fungus of a genus selected from Candida, Mucorales, Aspergillus, Cryptococcus, Histoplasma, and Pneumocystis.

17. The method of claim 14, wherein the infectious microbe is drug resistant.

18. The method of claim 17, wherein the infectious microbe is multi-drug resistant.
